(19) United States
(12) Patent Application Publication
(10) Pub. No.: US 2006/0058632 A1
(43) Pub. Date: Mar. 16, 2006

(54) Method of medical modeling

(76) Inventors: Doyle Heath McBurnett, Plano, TX (US); Tracy Claybrook McKissick, Garland, TX (US); Randall J. Smith, Plano, TX (US); Joshua C. Fagan, Denton, TX (US)

Correspondence Address: MICHAEL A. O'NEIL, P.C., 5949 SHERRY LANE, SUITE 820, DALLAS, TX, 75225, US

(21) Appl. No.: 10/939,572

(22) Filed: Sep. 13, 2004

(51) Int. Cl. A61B 5/05

(52) U.S. Cl. 600/410

(57) ABSTRACT

A method of medical modeling comprising the steps of identifying a plurality of medical facilities each having an MRI diagnostic system, providing a 3D printer at each of the identified medical facilities, conducting an MRI study at one of the identified medical facilities, transmitting MRI data comprising the MRI study from said one of the identified medical facilities to a processing compatible format at the processing center, transmitting the processed MRI data from a processing center to a 3D printer located on the premises of said one of the identified medical facilities, and utilizing the 3D printer to prepare a 3D model of the MRI study.

### TECHNICAL FIELD

This invention relates generally to the facilitation of surgical and other medical procedures, and more particularly to a method of converting MRI studies to 3D medical models.

### BACKGROUND AND SUMMARY OF THE INVENTION

Non-invasive examination of the human body began with the discovery of the x-ray. X-ray technology is extremely useful in determining the conditions of bones, teeth, and other solid and semi-solid components of the body, and is also useful in determining the presence of kidney stones, gallstones, etc. When used in conjunction with dyes and other media, x-ray investigations can also be useful in soft tissue studies.

More recently magnetic resonance imaging (MRI) technology has been introduced. MRI begins where x-ray leaves off in terms the examination of non-solid organs within the body particularly including muscles, tendons, etc. MRI studies are also useful in identifying and locating tumors and other soft tissue problems requiring treatment.

One difficulty associated with the use of MRI technology in the examination of soft tissues within the body involves the fact that MRI studies must be read by a radiologist. Thus, in most instances, the individual that is evaluating the MRI study is not the individual that is actually involved in treating the patient. As will therefore be apparent, a need exists for a system that allows treating physicians to take maximum advantage of MRI studies of the conditions that they are treating.

A seemingly unrelated technology is known as rapid prototyping. As is well known, computer assisted design (CAD) systems are now widely used in the design of machine components. Rapid prototyping involves the use of a 3D printer which receives dimension data directly from the CAD system and which converts the dimension data into a 3D prototype of the part of component that has been designed.

In accordance with the present invention MRI technology is combined with rapid prototyping technology to provide 3-dimensional (3D) models of MRI studies. The electronic information comprising a particular MRI study is directed to a facility wherein the MRI data is processed into the CAD data format. The processed MRI data is then directed to a 3D printer which prepares a 3D model of the MRI study.

More particularly, the present invention involves the use of a central processing facility to serve the needs of a plurality of hospitals and similar medical facilities. Each medical facility is equipped with at least one MRI diagnostic system and at least one 3D printer. At the conclusion of an MRI study the MRI data relating thereto is directed to the processing facility. At the processing facility the MRI data is processed into a 3D printer compatible format, typically a CAD data format. The processed MRI data is then directed to the 3D printer which is located at the same medical facility from which the MRI data was originally received. Upon receipt of the processed MRI data, the 3D printer proceeds to prepare a 3D model of the MRI study.

### BRIEF DESCRIPTION OF THE DRAWING

A more complete understanding of the present invention may be had by reference to the following Detailed Description when taken in connection with the accompanying Drawings, wherein:

FIG. 1 is a diagrammatic illustration of the method of the present invention;

FIG. 2 is a graphic illustration of the method of the present invention; and

FIG. 3 is a flowchart further illustrating the method of the present invention.

### DETAILED DESCRIPTION

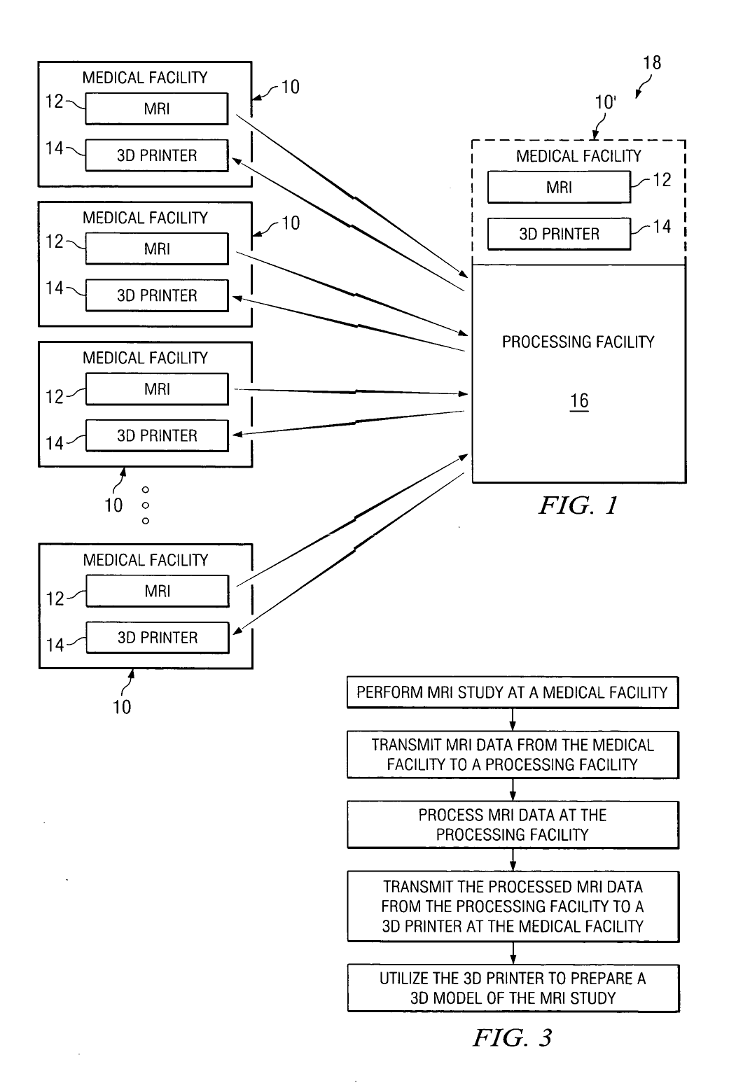

Referring to the Drawings, and particularly to FIG. 1 thereof, the present invention comprises a method of medical modeling wherein magnetic resonance imaging (MRI) studies are converted to 3D models for use by medical personnel particularly including treating physicians. In accordance with the invention a plurality of medical facilities 10 are each equipped with one or more MRI diagnostic systems 12. Each medical facility 10 is also provided with at least one 3D printer 14. At the completion of an MRI study at a particular medical facility 10, the results of the MRI study are electronically transmitted from the medical facility 10 to a processing facility 16.

The processing facility 16 converts the MRI data received from the medical facility 10 to a format or protocol which is compatible with the 3D printer located at the medical facility 10 at which the MRI study was originally conducted. The processed MRI data is then returned from the processing facility 16 to the medical facility 10 from which the MRI data was originally received. Upon receipt of the processed MRI data, the 3D printer at the medical facility prepares a 3D model corresponding to the results of the MRI study. The 3D model is then used by the medical personnel, particularly including treating physicians, for study and treatment of the medical condition comprising the subject matter of the MRI study.

As is indicated at 18, the processing facility 16 may be located within or immediately adjacent to a medical facility 10′ which is provided with one or more MRI systems 12 and at least one 3D printer 14. Although physically associated with the particular medical facility 10′, the processing facility 16 nevertheless provides processing services for a plurality of other medical facilities 10 in the manner described above.

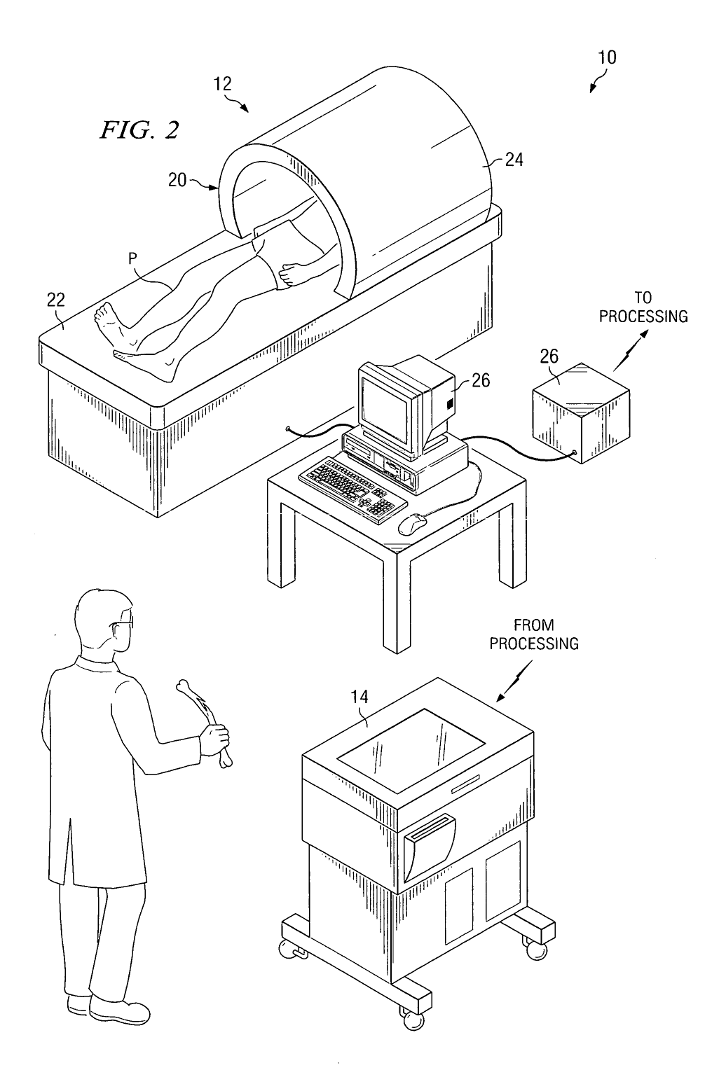

FIG. 2 illustrates the major physical components useful in the practice of the method of the present invention. An MRI diagnostic system 12 located within a medical facility 10 includes a conventional MRI machine 20 including a table 22 upon which a patient P being examined is located and an MRI chamber 24 which receives at least the components of the body of the patient P which will be examined during the MRI study. The MRI system 20 further includes a plurality of electronic components 26 which are used to control and monitor the operation of the MRI system 20. At the completion of a particular MRI study, the electronic components 26 direct the MRI data comprising the MRI study from the MRI system 12 to the processing facility 16 (FIG. 1).

The printer 14 which is utilized in the practice of the method of the present invention is also illustrated in FIG. 2. The printer 14 receives electronic signals comprising the MRI study that was previously performed utilizing the MRI system 20 from the processing facility 16 (FIG. 1) in a format or protocol which is compatible with the requirements of the printer 14, typically a CAD format. The printer 14 utilizes the received signals to prepare a 3D model of the MRI study.

The printer 14 may comprises a ZPrinter® 310 System or a z810 System both of which are available from Z Corporation, 20 North Avenue, Burlington, Mass. 01803. Alternatively, the printer 14 may comprise a 3D printer manufactured by 3D Systems Corporation, 26081 Avenue Hall, Valencia, Calif. 91355. Other 3D printers currently existing or hereinafter developed may also be utilized in the practice of the method of the present invention.

The method of the present invention is further illustrated in FIG. 3. The initial step in the method comprises performing an MRI study at a medical facility which may comprise a hospital, an emergency room, a surgical center, etc. MRI data comprising the MRI study is transmitted from the medical facility to a processing facility utilizing conventional data transmission techniques including but not limited to land lines, satellite and other wireless transmission facilities, and the internet, and other conventional facilities. At the processing facility, the data comprising the MRI study is converted from the MRI format to a format or protocol which is acceptable by the printer 14, typically a CAD data format.

Following the conversion step the processed MRI data is transmitted to a 3D printer which is located at the same medical facility wherein the MRI study was originally done. Again, the transmission of the MRI data to the 3D printer may be carried out utilizing land lines, satellite or other wireless facilities, the internet, or other conventional transmission facilities. Upon receipt of the processed MRI data the 3D printer functions to prepare a 3D model of the MRI study, that is, a 3D model of the component of the body of the patient P that was the subject matter of the MRI study.

The method of the present invention has a wide variety of utilizations. In the case of a broken bone, the method of the present invention provides a 3D model incorporating the exact location and nature of the break thereby allowing attending physicians to both formulate the best procedure for repairing the broken bone and to thereafter practice the selected procedure prior to the actual implementation thereof. In the case of a tumor or other growth which has established itself on a particular organ within the body of the patient, the method of the present invention can pinpoint the exact nature and location of the tumor or other growth as well as the location of the blood vessels which are supplying blood to the tumor or other growth thereby allowing attending physicians to formulate a procedure a effective treatment of the tumor or other growth. Other utilizations of the method of the present invention will readily suggest themselves to those skilled in the art.

Although preferred embodiments of the invention have been illustrated in the accompanying Drawings and described in the foregoing Detailed Description, it will be understood that the invention is not limited to the embodiments disclosed, but is capable of numerous rearrangements, modifications, and substitutions of parts and elements without departing from the spirit of the invention.

1. A method of medical modeling comprising the steps of:
identifying a plurality of medical facilities each having at least one MRI diagnostic system therein;
providing at least one 3D printer at each of the identified medical facilities;
providing a data processing facility;
conducting a MRI study at one of the identified medical facilities;
transmitting MRI data comprising the MRI study from said one of the identified medical facilities to the processing facility;
converting the MRI data from a first format to a second format at the processing facility and thereby providing processed MRI data;
transmitting the processed MRI data from the processing facility to a 3D printer located on the premises of said one of the identified medical facilities; and
utilizing the 3D printer to prepare a 3D model of the MRI study that was previously conducted at said one of the identified medical facilities.

2. The method of medical modeling according to claim 1 wherein each of the identified medical facilities is located at a geographical location which is substantially displaced from the geographical location of the remaining identified medical facilities.

3. The method of medical modeling according to claim 2 wherein data processing facility is situated at a geographical locations which is substantially separated from the geographical locations of each of the identified medical facilities.

4. The method of medical modeling according to claim 3 wherein at least one of the identified medical facilities is a hospital.

5. The method of medical modeling according to claim 3 wherein at least one of the identified medical facilities is an emergency medical facility.

6. The method of medical modeling according to claim 3 wherein at least one of the identified medical facilities comprising a surgical center.

7. The method of medical modeling according to claim 2 wherein the data processing facility is located within one of the identified medical facilities.

8. The method of medical modeling according to claim 2 wherein the data processing facility is located immediately adjacent to one of the identified medical modeling facilities.

9. The method of medical modeling according to claim 1 wherein the step of transmitting MRI data from said one of the identified medical modeling facilities to the data processing facility is carried out utilizing land lines.

10. The method of medical modeling according to claim 1 wherein the step of transmitting MRI data from said one of the identified medical modeling facilities to the processing facility is carried out utilizing wireless transmission facilities.

11. The method of medical modeling according to claim 1 wherein the step of transmitting MRI data comprising the MRI study from one of the identified medical modeling facilities to the processing facility is carried out utilizing the internet.

12. The method of medical modeling according to claim 1 wherein the step of transmitting processed MRI data from the data processing facility to said one of the identified medical modeling facilities to is carried out utilizing land lines.

13. The method of medical modeling according to claim 1 wherein the step of transmitting processed MRI data from the data processing facility to said one of the identified medical modeling facilities is carried out utilizing wireless transmission facilities.

14. The method of medical modeling according to claim 1 wherein the step of transmitting processed MRI data comprising the MRI study from the data processing facility to said one of the identified medical modeling facilities is carried out utilizing the internet.

15. The method of medical modeling according to claim 1 wherein the step of converting the MRI data from the first format to a second format at the processing facility is carried out by converting the MRI data from the format of the MRI diagnostic system of said one of the identified medical facilities to the format of the 3D printer located on the premises of said one of the medical facilities.

16. The method of medical modeling comprising the steps of:
identifying a plurality of medical facilities each having at least one MRI diagnostic system therein;
each of the identified medical facilities being located at a geographical location which is substantially displaced from the geographical location of the remaining identified medical facilities;
providing at least one 3D printer at each of the identified medical facilities;
providing a data processing facility;
conducting a MRI study at one of the identified medical facilities;
transmitting MRI data comprising the MRI study from said one of the identified medical facilities to the processing facility;
converting the MRI data from the format of the MRI diagnostic system located at said one of the identified medical facilities to the format of a 3D printer located at said one of the medical facilities at the processing facility and thereby providing processed MRI data;
transmitting the processed MRI data from the processing facility to the 3D printer located at said one of the identified facilities; and
utilizing the 3D printer to prepare a 3D model of the MRI study that was previously conducted at said one of the identified medical facilities.

17. The method of medical modeling according to claim 16 wherein the data processing facility of situated at a geographical location which is substantially displaced from the geographical locations of each of the identified medical facilities.

18. The method of medical modeling according to claim 16 wherein the data processing facility is located within one of the identified medical facilities.

19. The method of medical modeling according to claim 16 wherein the data processing facility is located immediately adjacent to one of the identified medical modeling facilities.

20. The method of medical modeling according to claim 16 wherein the step of transmitting MRI data comprising the MRI study from said one of the identified medical facilities to the data processing facility is carried out utilizing data transmission means selected from the group consisting of land lines, wireless transmission facilities, and the internet.

21. The method of medical modeling according to claim 16 wherein the step of transmitting the processed MRI data from the data processing facility to the 3D printer located facilities is carried out utilizing data transmission means selected from the group consisting of land lines, wireless transmission facilities, and the internet.
